(19) United States
(12) Patent Application Publication
(10) Pub. No.: US 2006/0099624 A1
(43) Pub. Date: May 11, 2006

(54) System and method for providing personalized healthcare for alzheimer's disease

(76) Inventors: Lu-yong Wang, Plainsboro, NJ (US); Amit Chakraborty, Cranbury, NJ (US); Dorin Comaniciu, Princeton Jct., NJ (US)

Correspondence Address: Siemens Corporation;Intellectual Property Department, 170 Wood Avenue South, Iselin, NJ, 08830, US

(21) Appl. No.: 11/249,184

(22) Filed: Oct. 13, 2005

Related U.S. Application Data
(60) Provisional application No. 60/619,786, filed on Oct. 18, 2004.

(51) Int. Cl. G06F 19/00; C12Q 1/68; G01N 33/53

(52) U.S. Cl. 435/6; 435/7.1; 702/19; 705/3

(57) ABSTRACT

A system and method for providing personalized health care for Alzheimer's disease (AD) are provided. A method for providing personalized healthcare to a patient suspect of having or having AD, includes: receiving heterogeneous data of the patient; fusing the heterogeneous data by using one of an information fusion or machine learning technique; and providing one of a diagnosis, prognosis or treatment for the patient based on the fused heterogeneous data.

### CROSS-REFERENCE TO RELATED APPLICATION

This application claims the benefit of U.S. Provisional Application No. 60/619,786, filed Oct. 18, 2004, the disclosure of which is herein incorporated by reference.

### BACKGROUND OF THE INVENTION

1. Technical Field

The present invention relates to providing personalized healthcare, and more particularly, to a system and method for providing personalized healthcare for Alzheimer's disease.

2. Discussion of the Related Art

Gene expression (also known as protein expression) is the process by which a gene's information is converted into the structures and functions of a cell. Gene expression is a multi-step process that begins with transcription and translation and is followed by folding, post-translation modification and targeting. The amount of protein that a cell expresses depends on the tisse, the developmental stage of the organism and the metabolic or physiologic state of the cell.

The expression of particular genes may be assessed with DNA microarray technology. DNA microarray technology can provide a rough measure of the cellular concentration of different mRNAs, often thousands at a time. A more sensitive and accurate method of relative gene expression measurement is a real-time polymerase chain reaction (PCR). With a carefully constructed standard curve it can produce an absolute measurement (e.g., in number of copies of mRNA per nanolitre of homogenized tissue, or in number of copies of mRNA per total poly-A RNA).

Genomic and proteomic techniques are increasingly being utilized to develop a variety of gene expression products for elucidating the molecular mechanism and pathogenesis of neurological diseases. Due to the diversity in cell types involved in neurological disease and the dynamic nature of gene and protein expression levels, it is compulsory to take into account temporal and spatial expression patterns when examining gene and protein expression profiles in the brain. However, the potential benefits of these products will not be fully appreciated until the molecular biology of certain neurological diseases is known.

Alzheimer's disease (AD) is a disease in which its molecular biology is still largely unknown. AD is a progressive neurodegenerative disorder and is one of the most common causes of dementia in the eldery and one of the leading causes of death in developed countries. AD is clinically characterized by progressive intellectual deterioration together with declining activities of daily living and neuropsychiatric symptoms or behavioral changes.

If an effort to find a cure for AD, its molecular mechanism has drawn much attention, but its pathogenesis is still largely undertermined. For example, it is still uncertain whether the central mechanism of AD neuro-degeneration is β-amyloid or neurofibrillary tangles (NFTs) of tau protein. In addition, AD's relationship with mitogen-activated protein kinase (MAPK), the apoptosis pathway, gene regulatory pathway and metabolic pathway problems and cytoskeletal, ubiqintin and cognitive impairment problems is still largely unknown.

By using high-throughput biotechnology such as DNA microarray and serial analysis of gene expression (SAGE), the pathology of AD is being uncovered and the treatment of AD is being enhanced. For example, microarray analysis has enabled the gene expression profile of AD to be retrieved. In addition, the comparison of global genomic mapping of the brain and medical imaging of the brain has been used to enhance the understanding of the structure and certain functions of AD.

One of the challenges for both genomic and proteomic techniques is making sense of the vast amounts of information generated thereby and utilizing this information for disease diagnosis, prognosis and treatment. Accordingly, there is a need for a technique of integrating a variety of diagnostic and treatment oriented platforms for enhancing the diagnosis, prognosis and treatment of AD.

### SUMMARY OF THE INVENTION

The present invention overcomes the foregoing and other problems encountered in the known teachings by providing a system and method for providing personalized healthcare for AD.

In one embodiment of the present invention, a method for providing personalized healthcare to a patient suspect of having or having AD, comprises: receiving heterogeneous data of the patient; fusing the heterogeneous data by using one of an information fusion or machine learning technique; and providing one of a diagnosis, prognosis or treatment for the patient based on the fused heterogeneous data.

The heterogeneous data comprises one or more of proteomic data of the patient, genomic data of the patient, medical imaging data of the patient, clinical data of the patient or epidimeological data of the patient.

The information fusion technique is a kernel-based information fusion technique. The machine learning technique is a kernel-based machine learning technique.

Providing one of a diagnosis, prognosis or treatment comprises: analyzing the fused heterogeneous data, wherein the fused heterogeneous data comprises genomic, proteomic or medical imaging data; and determining whether a tau protein or an amyloid beta induces MAPK.

Providing one of a diagnosis, prognosis or treatment comprises: injecting an amyloid into a brain of the patient; and identifying one of differently expressed genes, correlated genes, or apoptosis, metabolic, gene expression or regulatory pathways from the genomic data. A microarray analysis is performed on the genomic data.

The method further comprises identifying one of differently expressed genes, correlated genes, or apoptosis, metabolic, gene expression or regulatory pathways from the genomic data. The method further comprises identifying biomarkers based on the analysis of the genomic, proteomic or medical imaging data. The diagnosis indicates that the patient has AD or does not have AD or the patient has MCI or does not have MCI.

Providing one of a diagnosis, prognosis or treatment further comprises: analyzing the fused heterogeneous data, wherein the fused heterogeneous data comprises genomic, proteomic and medical imaging data; and determining an MCI molecular mechanism associated with the progression of MCI or AD or an MCI molecular mechanism inducing AD using the fused heterogeneous data.

Providing one of a diagnosis, prognosis or treatment further comprises identifying a putative MCI subtype based on a gene expression signature in gene expression data of the fused heterogeneous data, wherein the putative MCI subtype is identified by using a boosting tree.

In another embodiment of the present invention, a system for providing personalized healthcare to a patient suspect of having or having AD, comprises: a memory device for storing a program; a processor in communication with the memory device, the processor operative with the program code to: receive heterogeneous data of the patient; fuse the heterogeneous data, wherein the heterogeneous data is fused by using one of an information fusion or machine learning technique; and provide one of a diagnosis, prognosis or treatment for the patient based on the fused heterogeneous data.

The heterogeneous data comprises one or more of proteomic data of the patient, genomic data of the patient, medical imaging data of the patient, clinical data of the patient or epidimeological data of the patient.

The proteomic data is provided by a first high-throughput device, genomic data is provided by a second high-throughput device, medical imaging data is provided by an image acquisition device, clinical data is provided by a clinical database and epidimeological data is provided by an epidimeological database.

The information fusion technique is a kernel-based information fusion technique. The machine learning technique is a kernel-based machine learning technique.

The processor is further operative with the program code when providing one of a diagnosis, prognosis or treatment to: analyze the fused heterogeneous data, wherein the fused heterogeneous data comprises one of genomic, proteomic or medical imaging data; and determine whether a tau protein or an amyloid beta induces MAPK.

The processor is further operative with the program code when providing one of a diagnosis, prognosis or treatment to: identify one of differently expressed genes, correlated genes, or apoptosis, metabolic, gene expression or regulatory pathways from the genomic data after amyloid has been injected into the patient's brain.

The processor is further operative with the program code to: identify one of differently expressed genes, correlated genes, or apoptosis, metabolic, gene expression or regulatory pathways from the genomic data, when a microarray analysis is performed on the genomic data. The processor is further operative with the program code to: identify biomarkers based on the analysis of the genomic, proteomic or medical imaging data. The diagnosis indicates that the patient has AD or does not have AD or the patient has MCI or does not have MCI.

The processor is further operative with the program code when providing one of a diagnosis, prognosis or treatment to: analyze the fused heterogeneous data, wherein the fused heterogeneous data comprises genomic, proteomic and medical imaging data; and determine an MCI molecular mechanism associated with the progression of MCI or AD or an MCI molecular mechanism inducing AD using the fused heterogeneous data.

The processor is further operative with the program code when providing one of a diagnosis, prognosis or treatment to: identify a putative MCI subtype based on a gene expression signature in gene expression data of the fused heterogeneous data, wherein the putative MCI subtype is identified by using a boosting tree.

In yet another embodiment of the present invention, a method for database-guided decision support for providing personalized healthcare to a patient suspect of having or having AD, comprises: receiving medical imaging data of the patient from a medical imaging database; receiving genomic data of the patient from a genomic database; receiving proteomic data of the patient from a proteomic database; fusing the medical imaging, genomic and proteomic data by using one of an information fusion or machine learning technique; and determining a morphological association between AD and MCI.

The method further comprises: receiving clinical history data of the patient; and providing one of a diagnosis, prognosis or treatment for the patient based on the fused and clinical history data.

In another embodiment of the present invention, a database-guided decision support system for providing personalized healthcare to a patient suspect of having or having AD, comprises: an integrated database for providing heterogeneous data of the patient; and a fusion processor for receiving the heterogeneous data, fusing the heterogeneous data and providing one of a diagnosis, prognosis or treatment for the patient based on the fused heterogeneous data.

The heterogeneous data comprises one or more of proteomic data of the patient, genomic data of the patient, medical imaging data of the patient, clinical data of the patient or epidimeological data of the patient.

In yet another embodiment of the present invention, an integrated platform for analyzing microarray data for providing personalized healthcare to a patient having or suspect of having AD, comprises: a sampling module for receiving and processing microarray and medical imaging data of the patient; a visualization module for visualizing the processed microarray and medical imaging data; an analysis module for performing a classification analysis and a cluster analysis of the microarray and medical imaging data; and an annotation module for performing a first annotation based on the cluster analysis, a second annotation based on the classification analysis and a third annotation based on the visualized microarray and medical imaging data.

The foregoing features are of representative embodiments and are presented to assist in understanding the invention. It should be understood that they are not intended to be considered limitations on the invention as defined by the claims, or limitations on equivalents to the claims. Therefore, this summary of features should not be considered dispositive in determining equivalents. Additional features of the invention will become apparent in the following description, from the drawings and from the claims.

### BRIEF DESCRIPTION OF THE DRAWINGS

FIG. 1 is a diagram of a proposed molecular mechanism of AD;

FIG. 2 is a block diagram of a system for providing personalized healthcare for AD according to an exemplary embodiment of the present invention;

FIG. 3 is a block diagram of a fusion device according to an exemplary embodiment of the present invention;

FIG. 4 is a flowchart of a method for providing personalized healthcare for AD according to an exemplary embodiment of the present invention;

FIG. 5 is a block diagram of a system for database-guided decision support according to an exemplary embodiment of the present invention;

FIG. 6 is a flowchart of a method for database-guided decision support according to an exemplary embodiment of the present invention; and

FIG. 7 is a block diagram of a system for analyzing microarray data according to an exemplary embodiment of the present invention.

### DETAILED DESCRIPTION OF EXEMPLARY EMBODIMENTS

A system and method for providing personalized healthcare for AD according to an exemplary embodiment of the present invention provides an integrated healthcare approach by combining heterogeneous information such as phenotype and genotype information for the diagnosis, prognosis and treatment of AD. The system and method correlates genomic research with methods of clinical practice and research such as medical imaging to aid in the diagnosis, prognosis and treatment of AD. For example, the system and method combines phenotype and genotype data with information fusion and machine learning techniques to exploit uncertainties coming from both clinical and genomic fields.

The system and method also provides a database-guided decision support system based on heterogeneous information sources to assist in the diagnosis, prognosis and treatment of AD. The diagnosis process may involve, for example, employing advanced classification algorithms in the presence of phenotype and genotype uncertainties. The prognosis process may involve, for example, employing a time-course study using microarray at different stages of AD to understand the progressive mechanism thereof. The treatment process may involve, for example, analyzing change quantification problems arising from longitudinal studies before and after drug administration and pharmacogenomics studies for drug development.

Before discussing the exemplary embodiments of the present invention, a brief description of the proposed molecular mechanism of AD will be discussed followed by a brief overview of MCI and its subtypes.

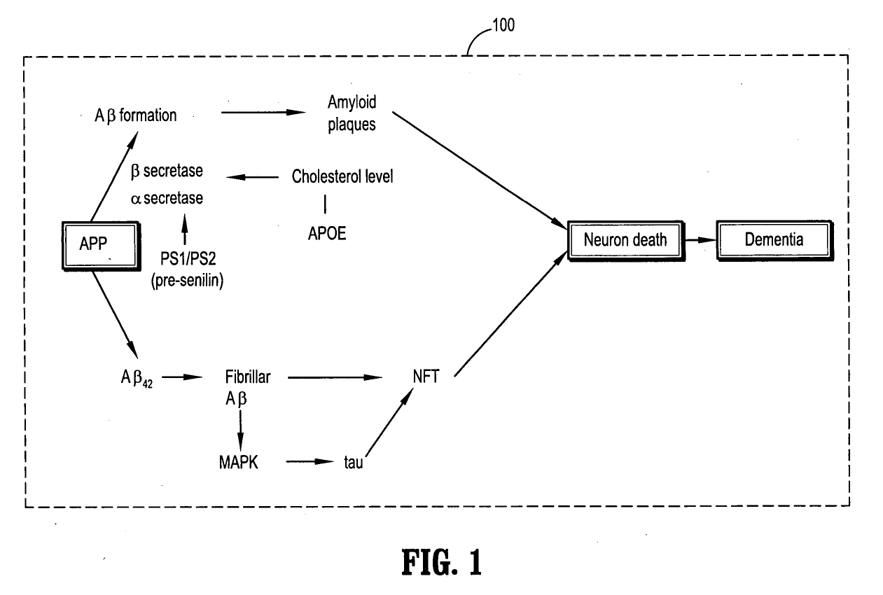

As shown by a diagram 100 of the proposed molecular mechanism of AD in FIG. 1, a vital event leading to AD (e.g., dementia) appears to be the formation of amyloid betas (Aβs). Amyloid betas cluster into amyloid plaques (e.g., senile plaques) on exterior surfaces of neurons and thereby lead to neuron death. An Aβ peptide is formed by an amyloid precursor protein (APP). There are two types of Aβ peptides: the 42 amino-acid amyloid beta peptide Aβ₄₂ and the 40 amino acid amyloid beta peptide Aβ₄₀. Fibrils of Aβ₄₂ have been shown to bundle together to form amyloid plaques.

Following amyloid plaque formation, two processes: inflammation and NFTs are believed to play a significant role in causing the death of a neuron. With regard to inflammation, two types of brain cells are involved in the immune/inflammatory response, they are: astrocytes and microglial. Astrocytes increase with the onset of AD and are activated to generate prostaglandin/arachidonic acid mediated inflammation. Activated microglial create damaging free radicals. The activities of astrocytes and microglial have been shown to lead to the death of neurons.

The tau protein (τ) is an essential protein that maintains the structural integrity of microtubules. In AD, however, the tau protein is hyper-phosphorylated and loses the capacity to bind to microtubules. The hyper-phosphorylated tau proteins bind to each other, wrapping themselves into knots with two threads of tau protein being wound around each other forming NFTs. Neurons full of NFTs rather than functional microtubules soon die. There is evidence that β-amyloid fibrils form pores in neurons leading to calcium influx and the neuron death associated with AD.

It is still undetermined whether the central mechanism of AD neuro-degeneration is β-amyloid or NFTs of tau protein. For example, it may be that the formation of amyloid plaques is an early event and that the formation of NFTs is a late event. The underlying processes of AD make each event seemingly independent. Based on previous experiments, amyloid plaques which were applied to cultured neurons and injected into the brains of non-human primates both led to NFTs. Further, fibrillar Aβ can induce MAPK to lead to tau phosphorylation and thus NFTs.

For example, MAPK pathways abnormally increase in AD, while they usually decrease with the aging of an immune system. Amyloid beta is always a feature of AD, but NFT is not. However, amyloid is not essential to cause the cell death of AD. Instead, tau has been shown to be essential for AD degeneration. Amyloid plaques typically appear in the association areas of the cerebral cortex, whereas NFTs usually begin in the entohinal cortex. NFTs develop more frequently in large pyramidal neurons with long cortical-cortical connections are associated with the origin of corticocortical projections whereas amyloid plaques are associated with the termination of corticocortical projections.

Mild cognitive impairment (MCI), which is a syndrome of memory impairment that does not significantly affect daily activities and is not accompanied by declines in overall cognitive function, has been identified as a potential transitional stage between normal aging and dementia. For example, research has found that between 6-25 percent of people with MCI progress to AD. Further, many experts have posited that MCI as well as typical age-related memory loss is an early form of AD and thus progression to symptomatic AD would eventually occur. Thus, MCI is becoming increasingly recognized as a risk factor for AD.

Table 1 illustrates several subtypes of MCI that are believed to represent prodromal stages for several dementing illnesses.

TABLE 1

| Type of MCI | May progress to: |
|---|---|
| Amnestic | AD |
| Multiple domains, mild impairment | AD |
| | Vascular dementia |
| | Dementia with Lewy bodies |
| | Normal aging |
| Single non-memory domain | Frontotemporal dementia |
| | Primary progressive aphasia |
| | Dementia with Lewy bodies |
| | Vascular dementia |

As shown in Table 1, MCI can affect a single cognitive memory or non-memory domain. In amnestic MCI, memory is affected to a significant degree (e.g., approximately 1.5 SD below age- and education-matched normal subjects), while other domains might be mildly impaired at perhaps less than 0.5 SD below appropriate comparison subjects. In multiple domain MCI, several cognitive domains are impaired at perhaps the 0.5-1.0 SD level of impairment. Single non-memory domain MCI is characterized by a person having a relatively isolated impairment in a single non-memory domain such as executive function, visuospatial processing or language.

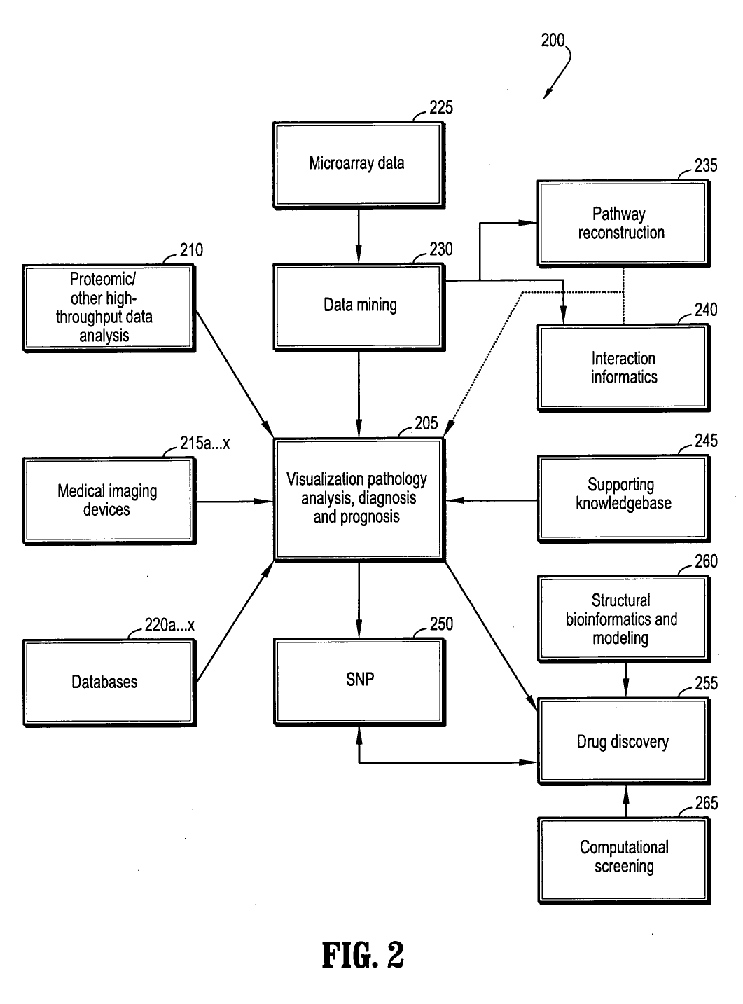

A system 200 for providing personalized healthcare for AD according to an exemplary embodiment of the present invention will now be described with reference to FIG. 2.

As shown in FIG. 2, a number of technologies such as proteomic and other high-throughput data analysis techniques such as two-dimensional polyacrylamide gel electrophoresis (2D-PAGE), mass spectrometry and medical imaging diagnosis are integrated by a fusion device 205 to diagnose, prognose and treat AD.

For example, a proteomic analysis device, processor or module 210 is coupled to the fusion device 205 for providing genetic information or analysis at organ, sub-cellular and molecular levels and for indicating intracellular processes in AD. The proteomic analysis device 210 may also provide information at the protein level to identify the molecular mechanism of AD. As further shown in FIG. 2, one or more medical imaging devices 215a . . . x may be connected to the fusion device 205.

An exemplary medical imaging device 215 may be, for example, image acquisition devices such as a magnetic resonance imaging (MRI) device, a computed tomography (CT) imaging device, a helical CT device, a positron emission tomography (PET) device, a 2D or three-dimensional (3D) fluoroscopic imaging device, a 2D, 3D, or four-dimensional (4D) ultrasound imaging device, or an x-ray device. The image acquisition device may also be a hybrid-imaging device capable of CT, MR, PET or other imaging techniques.

A microarray data source or sources 225 such as public databases, collaborating molecular biology labs or a local microarray analyzer is coupled to the fusion device 205 for providing microarray data thereto. The microarray data may be used to provide a snapshot of a genome-wide transcription profile and may be used by the fusion device 205 to identify cellular processes, transcription factors and their binding regions, gene interactions in the transcription process and genomic patterns for classification or relatedness tests.

The microarray data may also be mined by using a variety of data mining techniques available in a data mining processor or module 230 coupled to the fusion device 205. The data mining techniques are typically used to retrieve the gene expression profile of, for AD or MCI. Exemplary techniques for data mining include, inter alia, cluster analysis methods such as hierarchical, K-means, self-organizing map (SOM), principle component analysis (PCA) or mean-shift and classification methods such as support vector machine (SVM), decision tree, Bayesian classification and Fisher discriminatory analysis (FDA).

The fusion device 205 is also coupled to a pathway reconstruction processor or module 235 so that, for example, the analysis and identification of regulatory pathways for pathway reconstruction of common regulatory regions among strongly co-regulated genes may take place. A variety of techniques, tools or processes may be used for pathway reconstruction such as, for example, a gene microarray pathway profiler (GenMAPP), biological pathways exchange (BioPAX) and Reactome. When the pathway reconstruction processor or module 235 is coupled to an interaction informatics processor or module 240, the interaction information processor or module 240 may utilize a biomolecular interaction network database (BIND) to perform, for example, a protein analysis.

As further shown in FIG. 2, the fusion device 205 may also receive data from one or more databases 220a . . . x. An exemplary database 220 may include previous medical information of a patient such as prior CT scan information, gene expression data, treatment history, family history or demographics. In addition, a supporting knowledgebase 245 may be coupled to the fusion device 205 for providing information such as that associated with the relationships between genetic, clinical, medical imaging and other information. To accomplish this, the knowledgebase 245 may include genetic or proteomic reference data regarding AD, MCI and other diseases.

The fusion device 205 may also be coupled to drug discovery mechanisms 255-265 and a single nucleotide polymorphism (SNP) analyzer 250 that utilize genomic techniques to identify new gene targets for drug discovery and find associations between specific genetic markers and drug responses in a patient population. For example, since genome-wide searches for genes relevant to disease or therapy are used in conjunction with a polymorphism map distributed over a genome, SNPs may be used to provide relevance to a drug response. Thus, if a risk for a given disease is predicted to be high as judged by the SNP pattern of a patient, preventative therapy and lifestyle adjustments may be recommended by the fusion device 205 using the SNP analyzer 250.

By studying SNP profiles or haplotypes associated with traits of AD or MCI, relevant genes associated with AD or MCI may be identified and included, for example, in the knowledgebase 245. SNP association studies may also be used to indicate which pattern is most likely associated with disease causing genes. For example, the knowledgebase 245 may include associations between SNP profiling and common polygenic diseases, associations between SNPs and drug response, predicted molecular function changes from the structural context of missense mutation produced by cSNP and their relation with diseases such as AD or haplotyping and its relation to AD.

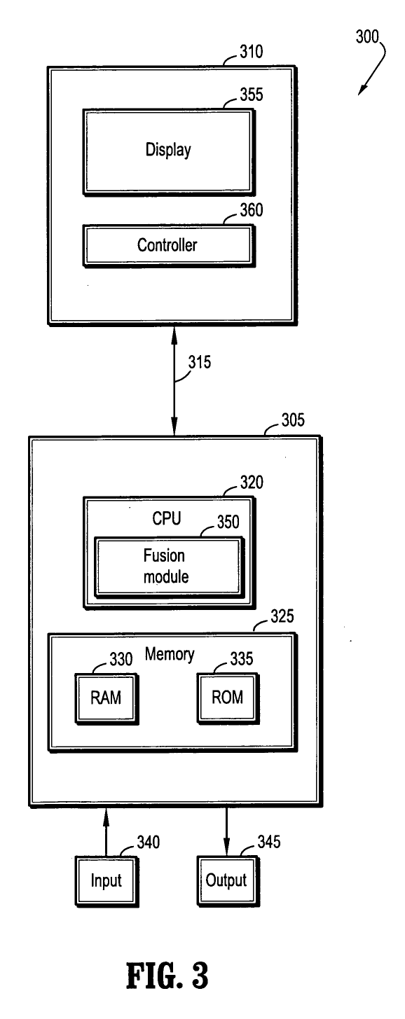

An exemplary fusion device 300 for use with the system 200 will now be described with reference to FIG. 3.

As shown in FIG. 3, the fusion device 300 includes, inter alia, a personal computer (PC) 305 and an operator's console 310 connected over, for example, an Ethernet network 315.

The PC 305, which may be a portable or laptop computer, a medical diagnostic imaging system or a picture archiving communications system (PACS) data management station, includes a CPU 320 and a memory 325, connected to an input device 340 and an output device 345. The CPU 320 also includes a fusion module 350 that includes one or more methods for fusing heterogeneous data of a patient by using an information fusion or machine learning technique for providing personalized healthcare for AD.

The memory 325 includes a random access memory (RAM) 330 and a read only memory (ROM) 335. The memory 325 can also include a database, disk drive, tape drive or a combination thereof. The memory 325 may be used to store, for example, genetics, clinical and medical imaging information such as genotype, gene, protein, polymorphisms, haplotypes or any combination thereof.

The RAM 330 functions as a data memory that stores data used during execution of a program in the CPU 320 and is used as a work area. The ROM 335 functions as a program memory for storing a program executed in the CPU 320. The input device 340 is constituted by a keyboard or mouse and the output device 345 is constituted by a liquid crystal display (LCD), cathode ray tube (CRT) display or printer.

The operation of the fusion device 300 is typically controlled from the operator's console 310, which includes a controller 360 such as a keyboard, and a display 355 such as a CRT display. The operator's console 310 may communicate with the PC 305 or any of the devices coupled to the system 300 so that, for example, 2D image data collected by the medical imaging devices 215a . . . x can be rendered into 3D data by the PC 305 and viewed on the display 355.

It is to be understood that the PC 305 can operate and display information provided by the medical imaging devices 215a . . . x absent the operator's console 310, using, for example, the input device 340 and output device 345 to execute certain tasks performed by the controller 360 and display 355.

The operator's console 310 may also communicate with any of the processors or modules coupled to the fusion device 205. For example, the operator's console 310 may be used to communicate with the microarray data source 225 such as a microarray analyzer to initiate the analysis of a DNA microarray. The operator's console 310 may then be used to cause the results of this analysis to be sent to one of the databases 220a . . . x for storage or to the PC 305 for further analysis.

The operator's console 310 may also include any suitable microarray image processing and analysis tool for measuring and visualizing proteomic or gene expression data. In addition, the operator's console 310 may include an image rendering system/tool/application that can process digital image data of an acquired image dataset (or portion thereof) to generate and display 2D and/or 3D images on the display 355. It is to be understood that the PC 305 may also include a microarray image processing and analysis tool for measuring and visualizing proteomic or gene expression data or an image rendering system/tool/application for processing digital image data of an acquired image dataset to generate and display 2D and/or 3D images.

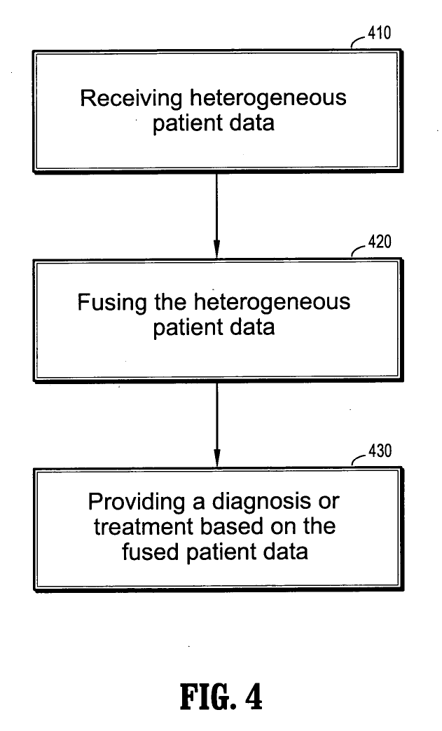

A method for providing personalized healthcare for AD according to an exemplary embodiment of the present invention will now be discussed with reference to FIG. 4.

As shown in FIG. 4, heterogeneous data associated with a patient is received, for example, by the fusion device 205 or 300 (410). The heterogeneous data may be, for example, proteomic data of the patient, genomic data of the patient, medical imaging data of the patient, clinical data of the patient, epidimeological data of the patient or any combination thereof. In addition, the heterogeneous data may be received from any of the processors or modules shown in FIG. 2, the operator's console 310 or a local or non-local singular or combinatory heterogeneous database.

Once the heterogeneous data is received, the heterogeneous data is fused, for example, by the fusion module 350 of the fusion device 300 (420). The heterogeneous data may be fused by employing an information fusion technique such as a kernel-based information fusion technique. For example, by using kernel-based information fusion, classifiers based on the heterogeneous data may be built by casting the heterogeneous data into a common format of kernel matrices. When using, for example, kernel-based machine learning techniques for fusion, a support vector machine may be employed for performing a discriminative diagnosis based on the heterogeneous data sources.

It is to be understood that by using kernel-based information fusion and learning techniques the heterogeneous data is represented by means of a kernel function. For example, the kernel function defines similarities between pairs of genes, proteins and so forth from the heterogeneous data. Each kernel function extracts a specific type of information from the heterogeneous data thereby providing a partial description or view of the data. Given many partial descriptions of the data, the descriptions are then combined using, for example, a semidefinite programming (SDP) method to yield sufficiently integrated or fused data. This data is then analyzed in conjunction with a support vector machine to provide decision support for diagnosis, prognosis or treatment of AD.

After the heterogeneous data has been fused, a diagnosis, prognosis or treatment based on the fused data is provided, for example, to the patient's doctor (430). The diagnosis may indicate that the patient has AD or does not have AD or that the patient has MCI or does not have MCI. The treatment such as a suggested course of treatment may be generated for the patient based on the diagnosis or the prognosis. The prognosis may indicate, for example, whether the patient has MCI and if their form of MCI includes AD-inducing MCI subtypes, thereby indicating that the patient has a greater risk of progressing to AD. A more detailed description of exemplary diagnosis, prognosis or treatment oriented procedures that may be performed prior to, during or after this step will now be described.

For example, in determining a diagnosis, prognosis or course of treatment for the patient based on their heterogeneous data such as genomic, proteomic or medical imaging data, it may be determined whether a tau protein or an amyloid beta induces MAPK. This may be done by using microarray and other proteomic tools or procedures. For example, an amyloid may be injected into the patient's brain and a microarray analysis may then be performed to identify differently expressed genes, correlated genes, or apoptosis, metabolic, gene expression or regulatory pathways. In addition, it may be determined what induces the hyper-phosphorylation of the tau protein, the relationship between the tau protein and amyloid beta and the molecular mechanism behind the relationship.

In another example, proteomic data of the patient may be analyzed to determine whether protein levels are varied due to a post-translation process. Biomarkers may be identified using CSF samples, blood or urine in combination with microarray or proteomic analysis or advanced analysis algorithms may be employed to classify patients having AD or not having AD or patients having MCI or not having MCI. In yet another example, the molecular mechanism of MCI associated with the progression of MCI or AD may be determined by using the fused heterogeneous data. Further, putative MCI subtypes based on gene expression signatures in gene expression data of the fused heterogeneous data may be identified by using boosting tree generated by a boosting method such as AdaBoost or Rankboost.

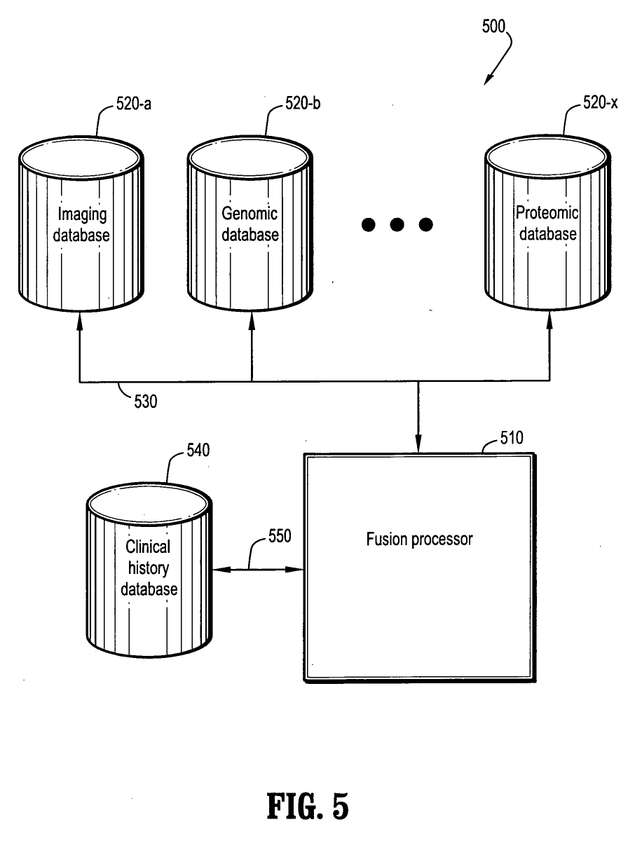

FIG. 5 is a block diagram of a system 500 for database-guided decision support according to an exemplary embodiment of the present invention.

As shown in FIG. 5, the system 500 for database-guided decision support includes a fusion system 510 coupled to a number of databases 520a, b . . . x via a network 530. The databases 520a, b . . . x may be, for example, an imaging database 520a, a genomic database 520b and a proteomic database 520x. It is to be understood, however, that the databases 520a, b . . . x can be any number or type of database such as those coupled to or part of the ancillary processors and modules coupled to the fusion device 205. In addition, the databases 520a, b . . . x can be separated as shown or integrated into a single database.

Further, the databases 520a, b . . . x can be local or non-local such as, for example, public microarray databases or those available from corroborating molecular biology laboratories. The fusion system 510 is also coupled to a clinical history database 540 via a network connection 550. The clinical history database 540 may include patient information such as prior imaging data, gene expression data, treatment history, family history or demographics and may be locally available such as those commonly found in a doctor's office or hospital.

It is to be understood that the fusion system 510 includes the same or similar components as the fusion devices 205 and 300 thus a description of its components will be omitted to avoid repetition. In addition, the fusion system 510 can be embodied as a fusion processor.

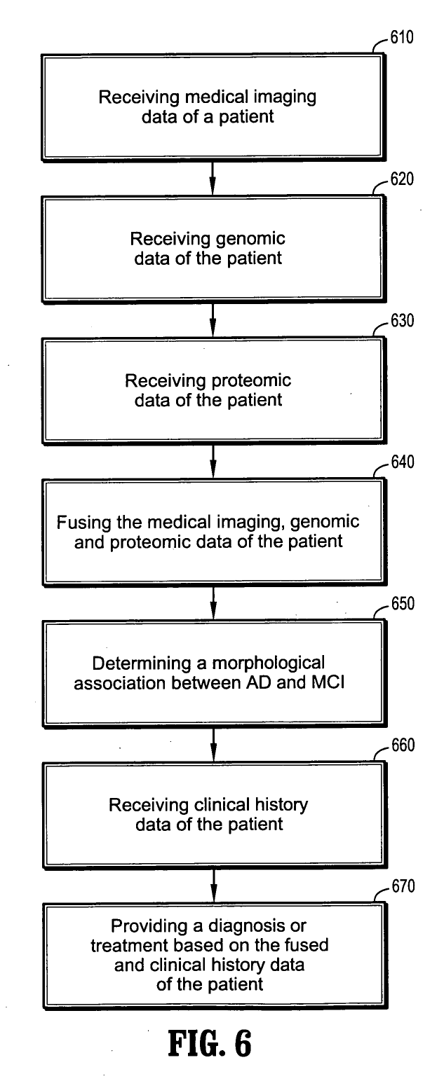

FIG. 6 is a flowchart of a method for database-guided decision support according to an exemplary embodiment of the present invention.

As shown in FIG. 6, medical imaging data of a patient is received, for example, by the fusion system 510 or the fusion devices 205 and 300 (610). For this example, we will refer only to the fusion system 510. Referring back to step 610, the medical imaging data from an acquisition device such as an MRI or CT scanner is provided from the imaging database 520a. Genomic data such as gene expression data or genotyping data of the patient from, for example, a microarray platform or an SNP chip is provided from the genomic database 520b to the fusion system 510 (620). Proteomic data of the patient from the proteomic database 520x is provided to the fusion system 510 (630).

Once the patient data has been received by the fusion system 510, it is fused using, for example, the kernel-based information fusion and machine learning techniques described above with reference to FIG. 4 (640). Using the fused information, a morphological association between AD and MCI is determined (650). The morphological association may be determined by utilizing some of the methods for diagnosis, prognosis and treatment discussed above with reference to FIG. 4. For example, the morphological association between AD and MCI may be used in conjunction with advanced cluster analysis algorithms and microarray data acquired over a period of time to elucidate the gene expression patterns of AD.

Upon determining the morphological association between AD and MCI, clinical history data of the patient is provided from the clinical history database 540 to the fusion system 510 (660). At this point, the clinical history data may also be fused or analyzed and used in conjunction with the fused data to provide a diagnosis, prognosis or course of treatment based thereon (670). This step may include the same or similar processes as described above for step 430. For example, a classification decision, which merges information encoded into various kernel matrices, may be used to obtain weights that reflect the relative importance of these information sources thereby enabling the fusion system 510 to provide a highly relevant and accurate diagnosis, prognosis or course of treatment for the patient.

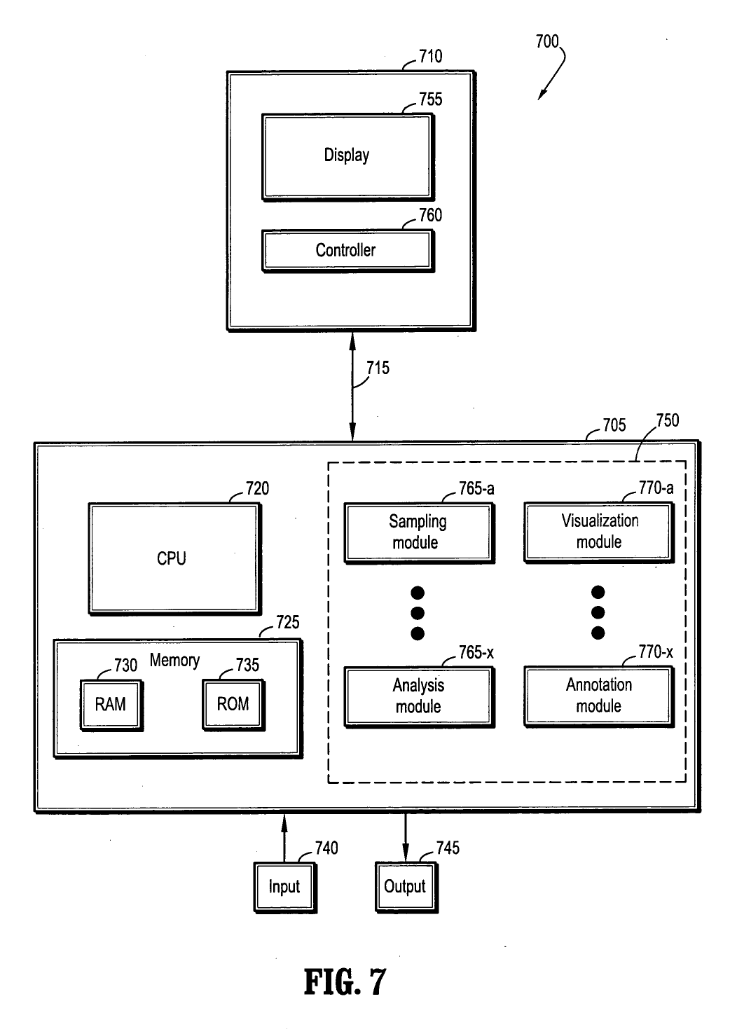

FIG. 7 is a block diagram of a system 700 for analyzing microarray imaging data that combines visualization, analysis, ontology annotation and pathway visualization into an integrated platform according to an exemplary embodiment of the present invention.

It is to be understood that the system 700 includes the same or similar components as those shown in FIG. 3 expect for an integration module 750, a sampling module 765a, analysis module 765x, visualization module 770a and annotation module 770x. As such, a description of the corresponding components will be omitted. It should be further understood that in an alternative embodiment the sampling module 765a, analysis module 765x, visualization module 770a and annotation module 770x may be included in the fusion device 205, the PC 305 and the fusion system 510.

As shown in FIG. 7, the sampling module 765a, analysis module 765x, visualization module 770a and annotation module 770x are included in the integration module 750. In addition to the procedures to be discussed below, integration module 750 is capable or performing the same or similar tasks as, for example, the fusion processor 305.

As further shown in FIG. 7, the sampling module 765a is configured to receive and process microarray data and medical image data. The visualization module 770a processes the microarray data and medical image data received from the sampling module 765a so that it may be viewed on a display device. The analysis module 765x then receives the output of the visualization module 770a and analyzes the microarray data and medical image data by utilizing cluster analysis or classification techniques. It should be understood that the analysis module 765x is capable of receiving, for example, genomic or proteomic data from other modules or externally coupled devices.

The annotation module 770x receives the outputs of the visualization module 770a and the analysis module 765x and provides an extensible markup language (XML)-based annotation to the received information so that it may be integrated with other types of XML data. For example, the annotation module 770x may provide XML-based pathway annotation to cluster analysis data provided by the analysis module 765x and XML-based gene ontology (GO) annotation to classification data also provided by the analysis module 765x so that this data can be integrated and viewed simultaneously on an output device and used to provide a diagnosis, prognosis or treatment plan based thereon. In addition, the annotation module 770x may provide a GenBank annotation based on the visualized microarray and medical imaging data.

It is to be understood that because some of the constituent system components and method steps depicted in the accompanying figures may be implemented in software, the actual connections between the system components (or the process steps) may differ depending on the manner in which the present invention is programmed. Given the teachings of the present invention provided herein, one of ordinary skill in the art will be able to contemplate these and similar implementations or configurations of the present invention.

It is to be further understood that the present invention may be implemented in various forms of hardware, software, firmware, special purpose processors, or a combination thereof. In one embodiment, the present invention may be implemented in software as an application program tangibly embodied on a program storage device (e.g., magnetic floppy disk, RAM, CD ROM, DVD, ROM, and flash memory). The application program may be uploaded to, and executed by, a machine comprising any suitable architecture.

It should also be understood that the above description is only representative of illustrative embodiments. For the convenience of the reader, the above description has focused on a representative sample of possible embodiments, a sample that is illustrative of the principles of the invention. The description has not attempted to exhaustively enumerate all possible variations. That alternative embodiments may not have been presented for a specific portion of the invention, or that further undescribed alternatives may be available for a portion, is not to be considered a disclaimer of those alternate embodiments. Other applications and embodiments can be straightforwardly implemented without departing from the spirit and scope of the present invention.

It is therefore intended that the invention not be limited to the specifically described embodiments, because numerous permutations and combinations of the above and implementations involving non-inventive substitutions for the above can be created, but the invention is to be defined in accordance with the claims that follow. It can be appreciated that many of those undescribed embodiments are within the literal scope of the following claims, and that others are equivalent.

What is claimed is:

1. A method for providing personalized healthcare to a patient suspect of having or having Alzheimer's disease (AD), comprising:
receiving heterogeneous data of the patient;
fusing the heterogeneous data by using one of an information fusion or machine learning technique; and
providing one of a diagnosis, prognosis or treatment for the patient based on the fused heterogeneous data.

2. The method of claim 1, wherein the heterogeneous data comprises one or more of proteomic data of the patient, genomic data of the patient, medical imaging data of the patient, clinical data of the patient or epidimeological data of the patient.

3. The method of claim 1, wherein the information fusion technique is a kernel-based information fusion technique.

4. The method of claim 1, wherein the machine learning technique is a kernel-based machine learning technique.

5. The method of claim 1, wherein providing one of a diagnosis, prognosis or treatment comprises:
analyzing the fused heterogeneous data, wherein the fused heterogeneous data comprises genomic, proteomic or medical imaging data; and
determining whether a tau protein or an amyloid beta induces mitogen-activated protein kinase (MAPK).

6. The method of claim 5, wherein providing one of a diagnosis, prognosis or treatment comprises:
injecting an amyloid into a brain of the patient; and
identifying one of differently expressed genes, correlated genes, or apoptosis, metabolic, gene expression or regulatory pathways from the genomic data.

7. The method of claim 5, wherein a microarray analysis is performed on the genomic data.

8. The method of claim 7, further comprising:
identifying one of differently expressed genes, correlated genes, or apoptosis, metabolic, gene expression or regulatory pathways from the genomic data.

9. The method of claim 5, further comprising:
identifying biomarkers based on the analysis of the genomic, proteomic or medical imaging data.

10. The method of claim 1, wherein the diagnosis indicates that the patient has AD or does not have AD or the patient has mild cognitive impairment (MCI) or does not have MCI.

11. The method of claim 1, wherein providing one of a diagnosis, prognosis or treatment further comprises:
analyzing the fused heterogeneous data, wherein the fused heterogeneous data comprises genomic, proteomic and medical imaging data; and
determining an MCI molecular mechanism associated with the progression of MCI or AD or an MCI molecular mechanism inducing AD using the fused heterogeneous data.

12. The method of claim 1, wherein providing one of a diagnosis, prognosis or treatment further comprises:
identifying a putative MCI subtype based on a gene expression signature in gene expression data of the fused heterogeneous data, wherein the putative MCI subtype is identified by using a boosting tree.

13. A system for providing personalized healthcare to a patient suspect of having or having Alzheimer's disease (AD), comprising:
a memory device for storing a program;
a processor in communication with the memory device, the processor operative with the program code to:
receive heterogeneous data of the patient;
fuse the heterogeneous data, wherein the heterogeneous data is fused by using one of an information fusion or machine learning technique; and
provide one of a diagnosis, prognosis or treatment plan for the patient based on the fused heterogeneous data.

14. The system of claim 13, wherein the heterogeneous data comprises one or more of proteomic data of the patient, genomic data of the patient, medical imaging data of the patient, clinical data of the patient or epidimeological data of the patient.

15. The system of claim 14, wherein the proteomic data is provided by a first high-throughput device, genomic data is provided by a second high-throughput device, medical imaging data is provided by an image acquisition device, clinical data is provided by a clinical database and epidimeological data is provided by an epidimeological database.

16. The system of claim 13, wherein the information fusion technique is a kernel-based information fusion technique.

17. The system of claim 13, wherein the machine learning technique is a kernel-based machine learning technique.

18. The system of claim 13, wherein the processor is further operative with the program code when providing one of a diagnosis, prognosis or treatment to:
analyze the fused heterogeneous data, wherein the fused heterogeneous data comprises one of genomic, proteomic or medical imaging data; and
determine whether a tau protein or an amyloid beta induces mitogen-activated protein kinase (MAPK).

19. The system of claim 18, wherein the processor is further operative with the program code when providing one of a diagnosis, prognosis or treatment to:
identify one of differently expressed genes, correlated genes, or apoptosis, metabolic, gene expression or regulatory pathways from the genomic data after amyloid has been injected into the patient's brain.

20. The system of claim 18, wherein the processor is further operative with the program code to:
identify one of differently expressed genes, correlated genes, or apoptosis, metabolic, gene expression or regulatory pathways from the genomic data, when a microarray analysis is performed on the genomic data.

21. The system of claim 18, wherein the processor is further operative with the program code to:
identify biomarkers based on the analysis of the genomic, proteomic or medical imaging data.

22. The system of claim 13, wherein the diagnosis indicates that the patient has AD or does not have AD or the patient has mild cognitive impairment (MCI) or does not have MCI.

23. The system of claim 13, wherein the processor is further operative with the program code when providing one of a diagnosis, prognosis or treatment to:
analyze the fused heterogeneous data, wherein the fused heterogeneous data comprises genomic, proteomic and medical imaging data; and
determine an MCI molecular mechanism associated with the progression of MCI or AD or an MCI molecular mechanism inducing AD using the fused heterogeneous data.

24. The system of claim 13, wherein the processor is further operative with the program code when providing one of a diagnosis, prognosis or treatment to:
identify a putative MCI subtype based on a gene expression signature in gene expression data of the fused heterogeneous data, wherein the putative MCI subtype is identified by using a boosting tree.

25. A method for database-guided decision support for providing personalized healthcare to a patient suspect of having or having Alzheimer's disease (AD), comprising:
receiving medical imaging data of the patient from a medical imaging database;
receiving genomic data of the patient from a genomic database;
receiving proteomic data of the patient from a proteomic database;
fusing the medical imaging, genomic and proteomic data by using one of an information fusion or machine learning technique; and
determining a morphological association between AD and mild cognitive impairment (MCI).

26. The method of claim 25, further comprising:
receiving clinical history data of the patient; and
providing one of a diagnosis, prognosis or treatment for the patient based on the fused and clinical history data.

27. A database-guided decision support system for providing personalized healthcare to a patient suspect of having or having Alzheimer's disease (AD), comprising:
an integrated database for providing heterogeneous data of the patient; and
a fusion processor for receiving the heterogeneous data, fusing the heterogeneous data and providing one of a diagnosis, prognosis or treatment for the patient based on the fused heterogeneous data.

28. An integrated platform for analyzing microarray data for providing personalized healthcare to a patient having or suspect of having Alzheimer's disease (AD), comprising:
a sampling module for receiving and processing microarray and medical imaging data of the patient;
a visualization module for visualizing the processed microarray and medical imaging data;
an analysis module for performing a classification analysis and a cluster analysis of the microarray and medical imaging data; and
an annotation module for performing a first annotation based on the cluster analysis, a second annotation based on the classification analysis and a third annotation based on the visualized microarray and medical imaging data.
